(19) United States
(12) Patent Application Publication
(10) Pub. No.: US 2020/0094213 A1
(43) Pub. Date: Mar. 26, 2020

(54) PROCESS FOR THE OLIGOMERIZATION OF ETHYLENE IN A COMPARTMENTALIZED GAS/LIQUID REACTOR

(71) Applicant: IFP Energies nouvelles, Rueil-Malmaison Cedex (FR)

(72) Inventors: Frederic AUGIER, Rueil-Malmaison Cedex (FR); Alexandre VONNER, Rueil-Malmaison Cedex (FR); Tiago SOZINHO, Rueil-Malmaison Cedex (FR); Natacha TOUCHAIS, Rueil-Malmaison Cedex (FR)

(73) Assignee: IFP Energies nouvelles, Rueil-Malmaison Cedex (FR)

(21) Appl. No.: 16/577,665

(22) Filed: Sep. 20, 2019

(30) Foreign Application Priority Data
Sep. 21, 2018 (FR) 18/58.608

(51) Int. Cl. B01J 10/00; B01J 23/755; C07C 2/08; B01J 19/00

(52) U.S. Cl. CPC B01J 10/007; B01J 23/755; C07C 11/08; B01J 19/0053; C07C 2/08

(57) ABSTRACT

Compartmentalized reactor which makes possible the oligomerization of olefins to give linear olefins and preferably to give linear α-olefins, comprising a reaction chamber and at least one heat exchanger(s). The compartmentalized reactor is also employed in an oligomerization process.

### TECHNICAL FIELD

The present invention relates to a compartmentalized reactor which makes possible the oligomerization of olefins to give linear olefins and preferably to give linear α-olefins, comprising a reaction chamber, compartmentalization means and at least one heat exchanger(s). The compartmentalized reactor is also employed in a process for the oligomerization of ethylene to give linear α-olefins, such as but-1-ene, hex-1-ene or oct-1-ene, or a mixture of linear α-olefins.

### PRIOR ART

The invention relates to the field of processes for the oligomerization, in particular for the dimerization, trimerization or tetramerization, of olefins to give linear olefins and more particularly to give linear α-olefins. The present invention applies to all the processes for the oligomerization of olefins, such as, for example, the trimerization of ethylene to give hex-1-ene, presented in the continuation of the description.

Typically, oligomerization processes are carried out in gas/liquid reactors, also known as bubble columns. Due to the exothermic nature of oligomerization reactions, bubble point reactors also comprise a loop for recirculation of a liquid fraction. The good heat transfer capacity related to the recirculation loop makes it possible to obtain a good homogeneity of the concentrations and to control the temperature throughout the reaction volume.

For a given operating temperature and a given operating pressure, the performance qualities of such a reactor, in terms of selectivity and of conversion, are limited by the kinetic scheme inherent to the catalytic system (the main and secondary reactions) and to the operating conditions under consideration (the temperature and the pressure).

The main oligomerization reactions correspond to the reactions for the dimerization, trimerization and tetramerization of the starting olefins to give final linear olefins, for example the conversion of ethylene to give hex-1-ene. The secondary reactions correspond to the reactions of the final linear olefins obtained during the main reactions, such as, for example, the reaction of hex-1-ene with ethylene to produce decenes. These secondary reactions result in a decrease in the yield of linear olefins in favor of non-upgradeable byproducts.

These byproducts associated with the operating conditions create a performance ceiling such as represented in the curve for selectivity as a function of the conversion (see FIG. 2A, described here in the case of the selective trimerization of ethylene to give hex-1-ene).

In particular, the processes of the prior art, employing a bubble point reactor, as illustrated in FIG. 1, do not make it possible to simultaneously achieve high levels of selectivity for linear olefins, more particularly for linear α-olefins, and high levels of conversion.

Surprisingly, the applicant company has discovered a specific implementation of the oligomerization process which makes it possible to simultaneously achieve higher levels of selectivity and of conversion than in the prior art. Such a process is carried out in a novel specific gas/liquid reactor comprising a reaction chamber comprising a plurality of compartmentalization means and at least one heat exchanger. Such a reactor makes it possible to approach a hydrodynamic behavior of a reactor of plug-flow type: the compartmentalization makes possible the segregation of the concentrations and the achievement of a homogeneous and laminar flow of the gas phase and of the liquid phase, greatly, indeed even completely, limiting the turbulent flow of the liquid phase which is typically encountered in the devices according to the prior art. Thus, the compartmentalization of the chamber of the reactor defines reaction zones with different concentrations of reaction liquid, thus making it possible to earn conversion points, with an unchanging selectivity, this being the case despite the exothermicity of the reaction. Thus, the oligomerization process according to the invention makes it possible to obtain an increase in the conversion of olefin(s), while retaining a virtually unchanging selectivity for linear olefins and in particular for α-olefins. These advantages make it possible to limit the costs for implementation of said process.

### SUBJECT MATTER OF THE INVENTION

The applicant company has developed a compartmentalized oligomerization reactor D having an upward stream of a liquid phase and of a gas phase forming a reaction medium, said reactor comprising:
a chamber (1) of elongated shape along the vertical axis with a height to width (H/W) ratio between 1 and 8;
means for introduction of a catalytic system (4) and of an olefin (3);
at least one heat exchanger (2) capable of cooling the reaction medium by means of a cooling liquid (6);
a means for recovery (7) of a liquid reaction effluent located in the final reaction zone (Zn) in the direction of flow of the liquid phase and of the gas phase;
a means for bleeding off (5) the gas phase, which gas phase is located at the top of said reactor (D);

characterized in that a plurality of compartmentalization means (8) are located inside the chamber (1) of said reactor (D), each compartmentalization means (8) extending radially over the entire section of the chamber (1) of said reactor, so as to form a plurality of reaction zones (Z1, Z2, . . . , Zn) laid out vertically in tiers, and in that each compartmentalization means (8) comprises a plurality of openings (12) with a diameter between 1 and 20 mm suitable for the passage of the liquid phase and of the gas phase from one reaction zone to the next, said plurality of openings (12) occupying between 3% and 60% of the total surface area of each compartmentalization means (8).

The applicant company has also discovered that said reactor can be employed in an olefin oligomerization process employing the reactor according to the invention, at a pressure between 1.0 and 10.0 MPa and at a temperature between 0° C. and 200° C., comprising the following stages:
a) the olefin and a catalytic oligomerization system comprising at least one metal precursor and at least one activating agent are introduced into the liquid phase of the reaction chamber 1;
b) said olefin and said system are brought into contact in each reaction zone Z1, Z2, . . . , Zn;
c) the reaction medium is cooled by means of at least one heat exchanger 2;
d) a liquid reaction effluent 7 is recovered in the upper part of the reaction chamber of the reactor.

### Definitions & Abbreviations

The following terms are defined in order to improve the understanding of the invention:

The term “oligomerization” denotes any addition reaction of a first olefin with a second olefin identical to or different from the first olefin and comprises dimerization, trimerization and tetramerization. The olefin thus obtained is of CₙH₂ₙ type, where n is equal to or greater than 4.

The term “olefin” denotes both an olefin and a mixture of olefins.

The term “α-olefin” denotes an olefin in which the double bond is located at the terminal position of the alkyl chain.

The term “heteroatom” is an atom other than carbon and hydrogen. A heteroatom can be chosen from oxygen, sulfur, nitrogen, phosphorus, silicon and halides, such as fluorine, chlorine, bromine or iodine.

The term “hydrocarbon” is an organic compound consisting exclusively of carbon (C) and hydrogen (H) atoms of empirical formula CₘHₚ, with m and p natural integers.

The term “catalytic system” denotes a mixture of at least one metal precursor, of at least one activating agent, optionally of at least one additive and optionally of at least one solvent.

The term “alkyl” is a saturated or unsaturated, linear or branched, non-cyclic, cyclic or polycyclic hydrocarbon chain comprising between 1 and 20 carbon atoms, preferably from 2 to 15 carbon atoms and more preferably still from 2 to 8 carbon atoms, denoted C₁-C₂₀ alkyl. For example, C₁-C₆ alkyl is understood to mean an alkyl chosen from the methyl, ethyl, propyl, butyl, pentyl, cyclopentyl, hexyl and cyclohexyl groups.

The term “aryl” is a fused or non-fused, mono- or polycyclic, aromatic group comprising between 6 and 30 carbon atoms, denoted C₆-C₃₀ aryl.

The term “alkoxy” is a monovalent radical consisting of an alkyl group bonded to an oxygen atom, such as the C₄H₉O— group.

The term “aryloxy” is a monovalent radical consisting of an aryl group bonded to an oxygen atom, such as the C₆H₅O— group.

The term “liquid phase” denotes the mixture of all the compounds which occur in the liquid physical state under the temperature and pressure conditions of the gas/liquid reactor.

The term “gas phase” denotes the mixture of all the compounds which occur in the gas physical state under the temperature and pressure conditions of the gas/liquid reactor: in the form of bubbles present in the liquid, and also in the top part of the gas/liquid reactor (also known as headspace of the reactor or gas headspace).

The term “lower part” of the reaction chamber of the compartmentalized gas/liquid reactor or of a reaction zone respectively denotes the lower half of the reactor or of the reaction zone.

The term “upper part” of the reaction chamber of the compartmentalized gas/liquid reactor or of a reaction zone respectively denotes the upper half of the reactor or of the reaction zone.

The term “withdrawal flow rate” denotes the weight of liquid withdrawn from the reactor per unit of time; it is expressed in tonnes per hour (t/h).

The term “non-condensable gas” denotes a byproduct resulting from the side reactions, in the gas physical form under the temperature and pressure conditions of the process, which accumulates in the headspace of the reactor. The non-condensable gases are, for example, ethane, methane or butane (non-exhaustive list).

The term “cocurrent” denotes the circulation of a first fluid in the same direction of circulation as a second fluid.

The term “exchange surface” represents the surface where heat exchanges take place between the reaction medium and the cooling liquid.

The term “solvent” denotes a liquid which has the property of dissolving, diluting or extracting other substances without chemically modifying them and without itself being modified. The expression “between . . . and . . . ” should be understood as including the limits mentioned.

### BRIEF DESCRIPTION OF THE FIGURES

The present invention is not limited to the implementations represented in the figures. The subject matter of the invention is illustrated in the figures through the specific case of the trimerization of ethylene to give hex-1-ene.

The figures do not represent all of the means necessary for the implementation of the reactors known to a person skilled in the art, such as the means for injection of the catalytic system, of the olefin, optionally of a solvent, the gas distributor, nor the means for control of the pressure and the temperature of the compartmentalized gas/liquid reactor. The subject matter of the present invention is not limited to the specific case of the trimerization of hex-1-ene, illustrated in the continuation of the description.

FIG. 1 illustrates a reactor according to the prior art, of bubble column type, comprising a reaction chamber 1′ with introduction of olefin via introduction means 3′. Withdrawal means 4′ make it possible, by virtue of a liquid recirculation pump 5′, to send a fraction of withdrawn liquid to a heat exchanger 2′ which makes it possible to remove the heat produced by the reaction and to feed, with cooled liquid, the top of the gas/liquid reactor via means for introduction of the cooled liquid 7′. The gas/liquid reactor comprises means for bleeding off 8′ the non-condensable gases in the gas headspace, in the upper part of the reactor, preferably at the top of the reactor. The effluent from the oligomerization process is recovered via the line 6′.

FIG. 2A is a diagram representing the selectivity for hex-1-ene as a function of the conversion of ethylene in a trimerization process according to the prior art (represented by points), comprising a gas/liquid reactor as represented in FIG. 1. The profile of the curve of FIG. 2A is substantially similar for all of the oligomerization reactions of olefins. It is important to note the difficulty in obtaining both a high level of conversion of ethylene (as % of ethylene converted) and a high selectivity for desired linear olefin(s) (as % by weight of the reaction products).

FIG. 2B is a diagram representing the selectivity for hex-1-ene as a function of the conversion of ethylene in a trimerization process according to the invention (represented by crosses) and according to the prior art (represented by points). Said process employs a compartmentalized gas/liquid reactor according to the invention comprising a heat exchanger positioned according to the present invention (i.e., at least one heat exchanger located inside or outside the reactor). The profile of the curve of FIG. 2B, obtained by the process according to the invention for the trimerization reaction of ethylene to give hex-1-ene, is representative of the technical effect of the invention, which is not limited to the trimerization. This is because this effect can be obtained for all oligomerization reactions of olefins and in particular dimerization and tetramerization reactions of ethylene.

FIGS. 2A and 2B represent the selectivity as a function of the conversion, with the selectivity, expressed as percentage, on the axis of the ordinates and the conversion, also expressed as percentage, on the axis of the abscissae.

FIG. 3 illustrates a compartmentalized gas/liquid reactor D, of bubble column type, according to a first embodiment of the invention, which makes possible the implementation of the process according to the invention and which comprises a reaction chamber 1, means for introduction of the catalytic system 4, fed with olefin(s) via introduction means 3, compartmentalization means 8 comprising a plurality of openings 12, located inside said chamber, extending radially over the entire section of the chamber, so as to form a plurality of reaction zones Z1, Z2, a heat exchanger 2 located inside said reactor, suitable for the cooling of the reaction medium and in which a cooling liquid 6 circulates, a means for bleeding the gas headspace 5 at the top of the reaction chamber and a means for recovery 7 of a reaction effluent in the upper part of said chamber.

FIG. 4 illustrates a compartmentalized gas/liquid reactor D, of bubble column type, according to a second embodiment of the invention, which makes possible the implementation of the process according to the invention, comprising five heat exchangers 2 outside the reaction chamber, each being incorporated in a recirculation loop and depending on a given reaction zone. Withdrawal means 9 make it possible, by virtue of a recirculation pump 10, to send the withdrawn liquid phase of the reaction medium to the heat exchanger 2, which makes it possible to remove the heat produced by the reaction and to feed, with cooled liquid, the reaction chamber 1, via introduction means 11.

### DETAILED DESCRIPTION OF THE INVENTION

Within the meaning of the present invention, the different embodiments presented can be used alone or in combination with one another, without any limit to the combinations. In the continuation of the description, the subject matter of the invention is illustrated in particular through the case of the trimerization of ethylene to give hex-1-ene.

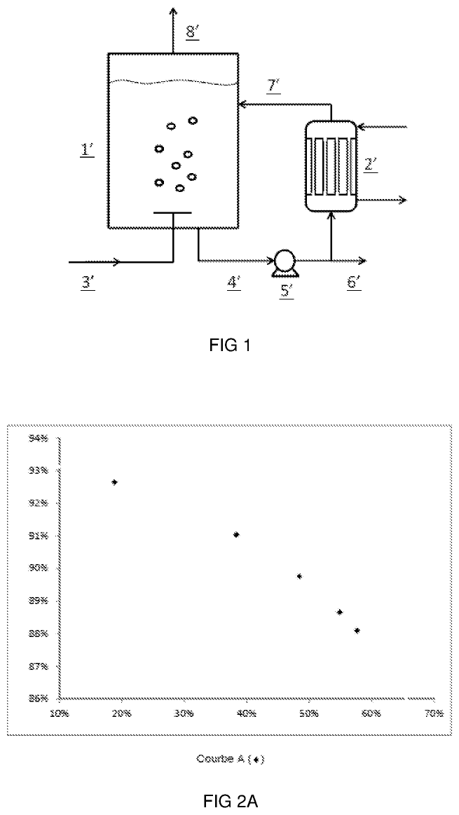

The applicant company has discovered that it is possible to improve the conversion of olefin(s), while retaining a high selectivity for desired linear olefin(s), and in particular α-olefin(s), by providing a specific device in the form of a compartmentalized gas/liquid reactor and at least one heat exchanger. Such a reactor makes it possible to approach a hydrodynamic behavior of a reactor of plug-flow type: the compartmentalization of the chamber makes possible the segregation of the concentrations and the achievement of a homogeneous and laminar flow of the gas phase and of the liquid phase, greatly, indeed even completely, limiting the turbulent flow of the liquid phase which is typically encountered in the devices according to the prior art (cf. FIG. 1). Thus, the compartmentalization of the chamber of the reactor defines reaction zones with different concentrations of reaction liquid, thus making it possible to earn conversion points, with an unchanging selectivity, this being the case despite the exothermicity of the reaction. Also, the presence of heat exchanger(s) limits the increase in temperature and makes possible the conversion of olefin(s) throughout the flow of the reaction medium, this being the case in each reaction zone.

The invention thus relates to a compartmentalized oligomerization reactor D having an upward stream of a liquid phase and of a gas phase forming a reaction medium, said reactor comprising:
a chamber (1) of elongated shape along the vertical axis with a height to width (H/W) ratio between 1 and 8;
means for introduction of a catalytic system (4) and of an olefin (3);
at least one heat exchanger (2) capable of cooling the reaction medium by means of a cooling liquid (6);
a means for recovery (7) of a liquid reaction effluent located in the final reaction zone (Zn) in the direction of flow of the liquid phase and of the gas phase;
a means for bleeding off (5) the gas phase, which gas phase is located at the top of said reactor (D);

characterized in that a plurality of compartmentalization means (8) are located inside the chamber (1) of said reactor (D), each compartmentalization means (8) extending radially over the entire section of the chamber (1) of said reactor, so as to form a plurality of reaction zones (Z1, Z2, . . . , Zn) laid out vertically in tiers, and in that each compartmentalization means (8) comprises a plurality of openings (12) with a diameter between 1 and 20 mm suitable for the passage of the liquid phase and of the gas phase from one reaction zone to the next, said plurality of openings (12) occupying between 3% and 60% of the total surface area of each compartmentalization means (8).

The invention also relates to an olefin oligomerization process employing the reactor according to the invention, at a pressure between 1.0 and 10.0 MPa and at a temperature between 0° C. and 200° C., comprising the following stages:
a) the olefin and a catalytic oligomerization system comprising at least one metal precursor and at least one activating agent are introduced into the liquid phase of the reaction chamber 1;
b) said olefin and said system are brought into contact in each reaction zone Z1, Z2, . . . , Zn;
c) the reaction medium is cooled by means of at least one heat exchanger 2;
d) a liquid reaction effluent 7 is recovered in the upper part of the reaction chamber of the reactor.

### Compartmentalized Gas/Liquid Reactor

The invention relates to a compartmentalized oligomerization reactor D having an upward stream of a liquid phase and of a gas phase forming a reaction medium, said reactor comprising:
a chamber (1) of elongated shape along the vertical axis with a height to width (H/W) ratio between 1 and 8;
means for introduction of a catalytic system (4) and of an olefin (3);
at least one heat exchanger (2) capable of cooling the reaction medium by means of a cooling liquid (6);
a means for recovery (7) of a liquid reaction effluent located in the final reaction zone (Zn) in the direction of flow of the liquid phase and of the gas phase;
a means for bleeding off (5) the gas phase, which gas phase is located at the top of said reactor (D);
   characterized in that a plurality of compartmentalization means (8) are located inside the chamber (1) of said reactor (D), each compartmentalization means (8) extending radially over the entire section of the chamber (1) of said reactor, so as to form a plurality of reaction zones (Z1, Z2, . . . , Zn) laid out vertically in tiers, and in that each compartmentalization means (8) comprises a plurality of openings (12) with a diameter between 1 and 20 mm suitable for the passage of the liquid phase and of the gas phase from one reaction zone to the next, said plurality of openings (12) occupying between 3% and 60% of the total surface area of each compartmentalization means (8).

Said reactor can also comprise a means for introduction of the olefin 3, located in the lower part of the reaction chamber, more particularly in the bottom of the chamber, employing a means for injection of the olefin within said liquid phase of the reaction chamber. Said reactor can also comprise a means for introduction of the catalytic system 4, located in the lower part, more particularly in the bottom of the reaction chamber.

According to the invention, the reaction chamber exhibits a height to width ratio (denoted H/W) between 1 and 8, preferably between 4 and 8. Preferably, the reaction chamber is of cylindrical shape.

The compartmentalized gas/liquid reactor comprises a means for bleeding off 5 the gas phase, which gas phase is located at the top of the reactor.

The compartmentalized gas/liquid reactor comprises a means for recovery 7 of a reaction effluent at the top of the chamber; preferably, the recovery means is located below the gas/liquid interface of the final reaction zone, in the direction of flow of the liquid phase and the gas phase.

Preferably, the gas/liquid reactor also comprises a pressure sensor which makes it possible to keep the pressure constant within the reaction chamber. Preferably, said pressure is kept constant by the introduction of additional olefin into the reaction chamber.

Preferably, the gas/liquid reactor also comprises a liquid level sensor; said level is kept constant by adjusting the flow rate of the effluent withdrawn in stage c) of the process according to the invention. Preferably, the level sensor is located at the interphase between the liquid phase and the gas headspace.

The compartmentalized gas/liquid reactor is preferably a gas/liquid/solid reactor, the solid phase comprising the catalyst.

### Compartmentalization Means

According to the invention, the compartmentalized gas/liquid reactor D comprises compartmentalization means 8 within the reaction chamber. Said means extend radially over the entire section of the chamber 1 of said reactor, so as to form a plurality of reaction zones Z1, Z2, . . . , Zn laid out vertically in tiers. The reaction zones are defined on the sides by the internal wall of the reaction chamber, above by the upper compartmentalization means or the roof of the chamber (for the final reaction zone Zn) and below by the lower compartmentalization means or the floor of the chamber (for the first reaction zone Z1). “n” is defined as a natural integer between 2 and 30, preferably between 2 and 20, more preferably between 2 and 15 and more preferably still between 4 and 10.

Preferably, the reaction zones all have the same volume.

Any compartmentalization means well known to a person skilled in the art can be used, such as a perforated plate.

Each compartmentalization means 8 comprises a plurality of openings 12 with a diameter between 1 and 20 mm, preferentially between 2 and 15 mm, preferably between 6 and 12 mm, suitable for the passage of the liquid phase and of the gas phase from one reaction zone to the next. Said plurality of openings 12 occupy between 3% and 60% of the total surface area of each compartmentalization means 8, preferentially between 20% and 60%, preferably between 35% and 55%.

Said means are capable of allowing the passage of the liquid phase and the gas phase of the reaction medium. Said compartmentalization means make it possible to approach a hydrodynamic behavior of a reactor of plug-flow type by segregating the concentrations and by greatly limiting, indeed even eliminating, the turbulent flow of the liquid phase, thus making it possible to have an upward laminar homogeneous liquid movement within the reaction chamber.

### Heat Exchanger

According to the invention, the compartmentalized gas/liquid reactor D comprises at least one heat exchanger(s) 2 in order to regulate the temperature within the reactor, in which a cooling liquid 6 circulates. Preferably, the cooling liquid circulates cocurrentwise with respect to the reaction medium.

The total surface area for exchange between the reaction medium, present within the chamber of said reactor, and the cooling liquid 6 is between 50 and 15 000 m². The surface area for exchange between the reaction medium and the cooling liquid, in each reaction zone, is between 2 and 8000 m².

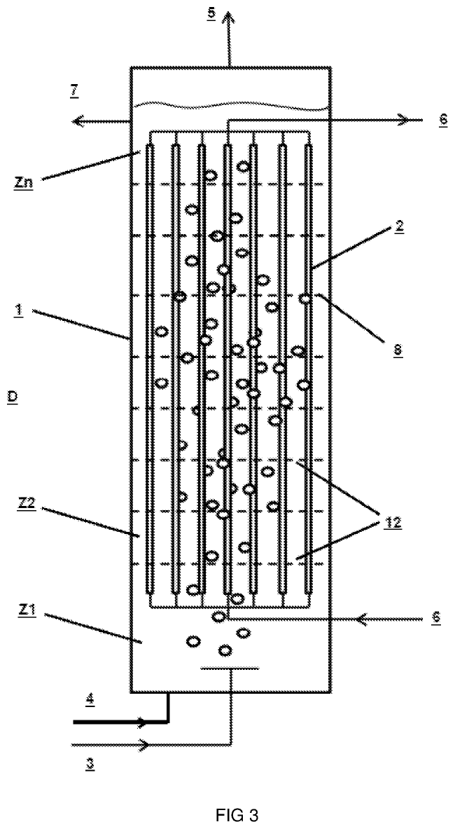

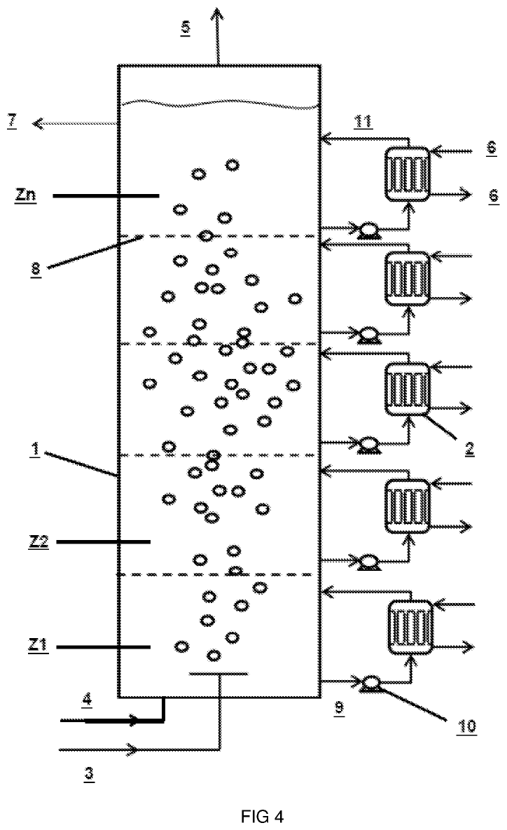

The heat exchangers suitable for cooling the liquid fraction are chosen from any means known to a person skilled in the art.
According to a first embodiment, the heat exchanger(s) are located inside the reaction chamber (FIG. 3). Preferably, said heat exchangers are positioned longitudinally with respect to the chamber of the reactor; preferentially, a heat exchanger is positioned in each reaction zone and more preferentially still a single heat exchanger is used inside said chamber.
According to a second embodiment, the heat exchanger(s) are located outside said reactor and each heat exchanger is incorporated in a recirculation loop comprising withdrawal means and means for introduction of the cooled liquid into the reaction chamber (FIG. 4).

Preferably, each reaction zone comprises a heat exchanger incorporated in a recirculation loop; preferentially, there are as many reaction zones as recirculation loops comprising a heat exchanger. Preferably, each reaction zone has its own recirculation loop with its point of entry of liquid and its point of departure of liquid originating from said loop. The recirculation loop can advantageously be implemented by any necessary means known to a person skilled in the art, such as a pump for the withdrawal of the liquid fraction, a means capable of regulating the flow rate of the withdrawn liquid fraction, or also a pipe for bleeding off at least a portion of the liquid fraction.

Preferably, the means for withdrawal of the liquid phase of the reaction medium from the chamber of the reactor is a pipe.

The heat exchanger(s) incorporated in the recirculation loop(s) make(s) possible good homogenization of the concentrations within each reaction zone and make it possible to control the temperature of the liquid phase of the reaction medium within the chamber.

The withdrawal means make it possible to send the withdrawn liquid to the heat exchanger. Thus, the heat produced by the reaction is removed and the withdrawn liquid is cooled in order to be introduced into the chamber via the introduction means.

For each reaction zone comprising a recirculation loop, the withdrawal of liquid from a given reaction zone is carried out starting from a point located below the point of introduction of the cooled liquid into said zone. For a given reaction zone, the withdrawal is preferably carried out in the lower part of the reaction zone.

For each reaction zone comprising a recirculation loop, the introduction of the cooled liquid into said reaction zone is carried out starting from a point located above the liquid withdrawal point. For a given reaction zone, the introduction is preferably carried out in the upper part of said zone.

For the heat exchanger of the final reaction zone Zn, in the direction of flow of the liquid phase and of the gas phase, the introduction of the cooled liquid is preferably carried out, into the gas phase, by any means known to a person skilled in the art.

For the heat exchanger of the first reaction zone Z1, in the direction of flow of the liquid phase and of the gas phase, the withdrawal is preferably carried out under the level of introduction of the olefin and preferentially in the bottom of the reaction chamber.

The withdrawal is carried out by any means capable of carrying out the withdrawal and preferably by using a pump.

The reaction mixture of said chamber is withdrawn by admission means under the control of the liquid level, so as to keep the latter constant. The admission means are any means well known to a person skilled in the art, such as a valve.

Advantageously, carrying out the cooling of the reaction medium via the recirculation loop also makes it possible to carry out the stirring of the medium and thus to homogenize the concentrations of the reactive entities throughout the liquid volume of the reaction chamber.

One advantage of the present invention is thus that of making it possible to achieve selectivities for linear olefins and preferably for linear α-olefins which are superior to those achieved with a reactor according to the prior art comprising only a single reaction chamber, this being obtained while retaining a high level of conversion into linear olefins and preferably into linear α-olefins.

### A Means for Introduction of the Olefin

According to the invention, the gas/liquid reactor D comprises a means for introduction 3 of the olefin, preferably located in the lower part of the reaction chamber, more particularly in the bottom of said chamber.

Preferably, the means for introduction of the olefin 3 is chosen from a pipe, a network of pipes, a multitubular distributor, a perforated plate or any other means known to a person skilled in the art.

Preferably, a gas distributor, which is a device which makes it possible to disperse the gas phase uniformly over the entire liquid section, is positioned at the end of the introduction means 3 within the chamber of the reactor. Said device comprises a network of perforated pipes, the diameter of the orifices of which is between 1 and 12 mm, preferably between 3 and 10 mm, in order to form ethylene bubbles in the liquid of millimetric size.

### A Means for Introduction of the Catalytic System

According to the invention, the compartmentalized gas/liquid reactor D comprises a means for introduction 4 of the catalytic system.

Preferably, the means for introduction of the catalytic system 4 is located in the lower part of the reaction chamber and preferably in the bottom of said chamber.

The means for introduction of the catalytic system 4 is chosen from any means known to a person skilled in the art and is preferably a pipe.

In the embodiment where the catalytic system is employed in the presence of a solvent or of a mixture of solvents, said solvent is introduced by an introduction means located in the lower part of the reaction chamber, preferably in the bottom of said chamber.

In one embodiment, the solvent can be introduced in one or more recirculation loops.

### Oligomerization Process

The process according to the invention makes it possible to obtain linear olefins and in particular linear α-olefins by bringing olefin(s) and a catalytic system into contact, optionally in the presence of an additive and/or of a solvent, and by the use of said compartmentalized gas/liquid reactor.

The oligomerization process is carried out at a pressure between 1.0 and 10.0 MPa, preferably between 2.0 and 8.0 MPa, more preferably between 4.0 and 8.0 MPa and more particularly between 6.0 and 8.0 MPa. The temperature is between 0° C. and 200° C., preferably between 30° C. and 180° C., more preferably between 30° C. and 150° C. and more preferably still between 40° C. and 140° C.

The residence time of the reaction medium in the reaction chamber is, on average, between 2 and 400 minutes, preferentially between 20 and 150 minutes, preferably between 30 and 120 minutes. The residence time of the reaction medium within each compartment is, on average, between 1 and 30 minutes, preferably between 5 and 20 minutes and more preferably still between 5 and 15 minutes.

### Stage a) of Introduction of the Olefin and of the Catalytic System

The process according to the invention comprises a stage a) of introduction of the olefin and of the catalytic system comprising at least one metal precursor and at least one activating agent into the liquid phase of the gas/liquid reactor D.

### The Olefin

The process according to the invention can comprise the introduction of olefin or of a mixture of olefins. Preferably, the olefin is ethylene.

The olefin is introduced by dispersion in the liquid phase of the compartmentalized gas/liquid reactor, preferably in the lower part of the compartmentalized gas/liquid reactor, more preferably in the compartment Z1 and more particularly in the bottom of the reaction chamber.

The olefin can be introduced into each reaction zone of the chamber of the compartmentalized gas/liquid reactor, more preferably into the reaction zones located in the lower part of said chamber. More particularly, when the olefin is introduced into a reaction zone, the introduction is carried out in the lower part of said zone.

In one embodiment, the olefin can be introduced in one or more recirculation loops.

Preferably, the olefin is introduced by a means capable of producing said dispersion uniformly over the entire section of the reaction chamber. Preferably, the dispersion means is chosen from a distributing system with a homogeneous distribution of the points for introduction of the olefin over the entire section of said chamber.

The olefin is introduced by at least one means for admission under the control of the pressure, which keeps the latter constant in the reactor. The admission means is any means well known to a person skilled in the art, such as a valve.

Preferably, the olefin is introduced at a flow rate between 1 and 200 t/h, preferably between 3 and 150 t/h, preferably between 5 and 100 t/h and preferably between 5 and 50 t/h.

According to a specific embodiment of the invention, a stream of gaseous hydrogen can also be introduced into the reaction chamber, with a flow rate representing from 0.2% to 1.0% by weight of the flow rate of olefin introduced. Preferably, the stream of gaseous hydrogen is introduced by the means employed for the introduction of the olefin.

According to one embodiment, the catalytic oligomerization reaction is carried out continuously and in homogeneous catalysis, in the absence of support. The olefin can be introduced just as easily via the means for introduction of the catalytic system as independently.

Preferably, the velocity of the olefin at the outlet of the orifices is between 1 and 30 m/s. Its superficial velocity (gas volumetric velocity divided by the section of the gas/liquid reactor) is between 0.5 and 10 cm/s and preferably between 1 and 8 cm/s.

### The Catalytic System

According to one embodiment, the catalytic system is introduced into the lower part of the compartmentalized gas/liquid reactor, more preferably into the compartment Z1 and more particularly into the bottom of the reaction chamber.

In one embodiment, the catalytic system can be introduced in one or more recirculation loops.

Any catalytic system known to a person skilled in the art and capable of being employed in the dimerization, trimerization or tetramerization processes and more generally in the oligomerization processes according to the invention comes within the field of the invention. Said catalytic systems and also their implementations are described in particular in the applications FR 2 984 311, FR 2 552 079, FR 3 019 064, FR 3 023 183, FR 3 042 989 or also in the application FR 3 045 414.

Preferably, the catalytic systems comprise, preferably consist of:
a metal precursor, preferably based on nickel, on titanium or on chromium,
an activating agent,
optionally an additive, and
optionally a solvent.

### The Metal Precursor

The metal precursor used in the catalytic system is chosen from compounds based on nickel, on titanium or on chromium.

In one embodiment, the metal precursor is based on nickel and preferably comprises nickel with a (+II) oxidation state. Preferably, the nickel precursor is chosen from nickel(II) carboxylates, such as, for example, nickel 2-ethylhexanoate, nickel(II) phenates, nickel(II) naphthenates, nickel(II) acetate, nickel(II) trifluoroacetate, nickel(II) triflate, nickel(II) acetylacetonate, nickel(II) hexafluoroacetylacetonate, π-allylnickel(II) chloride, π-allylnickel(II) bromide, methallylnickel(II) chloride dimer, η³-allylnickel(II) hexafluorophosphate, η³-methallylnickel(II) hexafluorophosphate and nickel(II) 1,5-cyclooctadienyl, in their hydrated or nonhydrated form, taken alone or as a mixture.

In a second embodiment, the metal precursor is based on titanium and preferably comprises a titanium aryloxy or alkoxy compound.

The titanium alkoxy compound advantageously corresponds to the general formula [Ti(OR)₄] in which R is a linear or branched alkyl radical. Mention may be made, among the preferred alkoxy radicals, as nonlimiting examples, of tetraethoxy, tetraisopropoxy, tetra(n-butoxy) and tetra(2-ethylhexyloxy).

The titanium aryloxy compound advantageously corresponds to the general formula [Ti(OR′)₄] in which R′ is an aryl radical substituted or unsubstituted by alkyl or aryl groups. The R′ radical can comprise heteroatom-based substituents. The preferred aryloxy radicals are chosen from phenoxy, 2-methylphenoxy, 2,6-dimethylphenoxy, 2,4,6-trimethylphenoxy, 4-methylphenoxy, 2-phenylphenoxy, 2,6-diphenylphenoxy, 2,4,6-triphenylphenoxy, 4-phenylphenoxy, 2-(tert-butyl)-6-phenylphenoxy, 2,4-di(tert-butyl)-6-phenylphenoxy, 2,6-diisopropylphenoxy, 2,6-di(tert-butyl)phenoxy, 4-methyl-2,6-di(tert-butyl)phenoxy, 2,6-dichloro-4-(tert-butyl)phenoxy and 2,6-dibromo-4-(tert-butyl)phenoxy, the biphenoxy radical, binaphthoxy or 1,8-naphthalenedioxy.

According to a third embodiment, the metal precursor is based on chromium and preferably comprises a chromium(II) salt, a chromium(III) salt or a salt with a different oxidation state which can comprise one or more identical or different anions, such as, for example, halides, carboxylates, acetylacetonates or alkoxy or aryloxy anions. Preferably, the chromium-based precursor is chosen from CrCl₃, CrCl₃(tetrahydrofuran)₃, Cr(acetylacetonate)₃, Cr(naphthenate)₃, Cr(2-ethylhexanoate)₃ or Cr(acetate)₃.

The concentration of nickel, of titanium or of chromium is between 0.01 and 300.0 ppm by weight of atomic metal, with respect to the reaction mass, preferably between 0.02 and 100.0 ppm, preferentially between 0.03 and 50.0 ppm, more preferentially between 0.5 and 20.0 ppm and more preferentially still between 1.0 and 20.0 ppm by weight of atomic metal, with respect to the reaction mass.

### The Activating Agent

Whatever the metal precursor, the catalytic system additionally comprises one or more activating agents chosen from aluminum-based compounds, such as methylaluminum dichloride (MeAlCl₂), dichloroethylaluminum (EtAlCl₂), ethylaluminum sesquichloride (Et₃Al₂Cl₃), chlorodiethylaluminum (Et₂AlCl), chlorodiisobutylaluminum (i-Bu₂AlCl), triethylaluminum (AlEt₃), tripropylaluminum (Al(n-Pr)₃), triisobutylaluminum (Al(i-Bu)₃), diethylethoxyaluminum (Et₂AlOEt), methylaluminoxane (MAO), ethylaluminoxane and modified methylaluminoxanes (MMAO).

### The Additive

Optionally, the catalytic system comprises one or more additives.

When the catalytic system is based on nickel, the additive is chosen from:
compounds of nitrogenous type, such as trimethylamine, triethylamine, pyrrole, 2,5-dimethylpyrrole, pyridine, 2-methylpyridine, 3-methylpyridine, 4-methylpyridine, 2-methoxypyridine, 3-methoxypyridine, 4-methoxypyridine, 2-fluoropyridine, 3-fluoropyridine, 3-trifluoromethylpyridine, 2-phenylpyridine, 3-phenylpyridine, 2-benzylpyridine, 3,5-dimethylpyridine, 2,6-di(tert-butyl)pyridine and 2,6-diphenylpyridine, quinoline, 1,10-phenanthroline, N-methylpyrrole, N-butylpyrrole, N-methylimidazole, N-butylimidazole, 2,2′-bipyridine, N,N′-dimethylethane-1,2-diimine, N,N′-di(t-butyl)ethane-1,2-diimine, N,N′-di(t-butyl)butane-2,3-diimine, N,N′-diphenylethane-1,2-diimine, N,N′-bis(2,6-dimethylphenyl)ethane-1,2-diimine, N,N′-bis(2,6-diisopropylphenyl)ethane-1,2-diimine, N,N′-diphenylbutane-2,3-diimine, N,N′-bis(2,6-dimethylphenyl)butane-2,3-diimine or N,N′-bis(2,6-diisopropylphenyl)butane-2,3-diimine, or
compounds of phosphine type independently chosen from tributylphosphine, triisopropylphosphine, tricyclopentylphosphine, tricyclohexylphosphine, triphenylphosphine, tris(o-tolyl)phosphine, bis(diphenylphosphino)ethane, trioctylphosphine oxide, triphenylphosphine oxide or triphenyl phosphite, or
compounds corresponding to the general formula (I) or one of the tautomers of said compound:

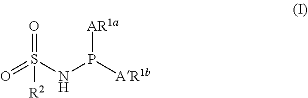

in which:
A and A′, which are identical or different, are independently an oxygen or a single bond between the phosphorus atom and a carbon atom,
the R^{1a} and R^{1b} groups are independently chosen from the methyl, trifluoromethyl, ethyl, n-propyl, isopropyl, n-butyl, isobutyl, t-butyl, pentyl, cyclohexyl or adamantyl groups, which are substituted or unsubstituted and contain or do not contain heteroelements; the phenyl, o-tolyl, m-tolyl, p-tolyl, mesityl, 3,5-dimethylphenyl, 4-(n-butyl)phenyl, 2-methylphenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-di(tert-butyl)-4-methoxyphenyl, 4-chlorophenyl, 3,5-di(trifluoromethyl)phenyl, benzyl, naphthyl, binaphthyl, pyridyl, bisphenyl, furanyl or thiophenyl groups,
the R² group is independently chosen from the methyl, trifluoromethyl, ethyl, n-propyl, isopropyl, n-butyl, isobutyl, t-butyl, pentyl, cyclohexyl or adamantyl groups, which are substituted or unsubstituted and contain or do not contain heteroelements; the phenyl, o-tolyl, m-tolyl, p-tolyl, mesityl, 3,5-dimethylphenyl, 4-(n-butyl)phenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-di(tert-butyl)-4-methoxyphenyl, 4-chlorophenyl, 3,5-bis(trifluoromethyl)phenyl, benzyl, naphthyl, binaphthyl, pyridyl, bisphenyl, furanyl or thiophenyl groups.

When the catalytic system is based on titanium, the additive is chosen from diethyl ether, diisopropyl ether, dibutyl ether, diphenyl ether, 2-methoxy-2-methylpropane, 2-methoxy-2-methylbutane, 2,2-dimethoxypropane, 2,2-di(2-ethylhexyloxy)propane, 2,5-dihydrofuran, tetrahydrofuran, 2-methoxytetrahydrofuran, 2-methyltetrahydrofuran, 3-methyltetrahydrofuran, 2,3-dihydropyran, tetrahydropyran, 1,3-dioxolane, 1,3-dioxane, 1,4-dioxane, dimethoxyethane, di(2-methoxyethyl) ether, benzofuran, glyme and diglyme, taken alone or as a mixture.

When the catalytic system is based on chromium, the additive is chosen from:
aryloxy compounds of general formula [M(R³O)₂₋ₙXₙ]_{y}, in which:
   M is chosen from magnesium, calcium, strontium and barium, preferably magnesium,
   R³ is an aryl radical containing from 6 to 30 carbon atoms and X is a halogen or an alkyl radical containing from 1 to 20 carbon atoms,
   n is an integer which can take the values of 0 or 1, and
   y is an integer between 1 and 10; preferably, y is equal to 1, 2, 3 or 4.

Preferably, the aryloxy radical R³O is chosen from 4-phenylphenoxy, 2-phenylphenoxy, 2,6-diphenylphenoxy, 2,4,6-triphenylphenoxy, 2,3,5,6-tetraphenylphenoxy, 2-(tert-butyl)-6-phenylphenoxy, 2,4-di(tert-butyl)-6-phenylphenoxy, 2,6-diisopropylphenoxy, 2,6-dimethylphenoxy, 2,6-di(tert-butyl)phenoxy, 4-methyl-2,6-di(tert-butyl)phenoxy, 2,6-dichloro-4-(tert-butyl)phenoxy and 2,6-dibromo-4-(tert-butyl)phenoxy. The two aryloxy radicals can be carried by one and the same molecule, such as, for example, the biphenoxy radical, binaphthoxy or 1,8-naphthalenedioxy. Preferably, the aryloxy radical R³O is 2,6-diphenylphenoxy, 2-(tert-butyl)-6-phenylphenoxy or 2,4-di(tert-butyl)-6-phenylphenoxy.

### The Solvent

In another embodiment according to the invention, the catalytic system optionally comprises one or more solvents.

The solvent is chosen from the group formed by aliphatic and cycloaliphatic hydrocarbons, such as hexane, cyclohexane, heptane, butane or isobutane.

Preferably, the solvent used is cyclohexane.

Stage b) of Bringing the Olefin and the Catalytic System into Contact

The olefin and the catalytic system are brought into contact in each reaction zone Z1, Z2, . . . , Zn. This time over which the olefin and the catalytic system are brought into contact in each reaction zone Z1, Z2, . . . , Zn is between 0.5 and 30 seconds, preferably between 1 and 20 seconds and more preferably still between 1 and 15 seconds.

### Stage c) of Cooling the Reaction Medium

The process according to the invention comprises a stage c) of cooling the reaction medium. The reaction medium present within the reaction chamber of the compartmentalized gas/liquid reactor is cooled by means of at least one heat exchanger.

As the reaction is exothermic, it is necessary to remove the heat produced by the reaction by cooling the reaction medium in order to control the temperature in the whole of the chamber of the reactor and thus to make possible the progression of the reaction.

Preferably, said stage consisting in cooling the reaction medium is carried out by the presence of at least one heat exchanger(s), inside or outside the chamber of the reactor, and preferably located inside. More preferably, a single heat exchanger is used and placed inside the chamber of the reactor.

The presence of at least one heat exchanger(s), in which a cooling liquid circulates, advantageously makes it possible to reduce the temperature of the reaction medium by 1.0° C. to 11.0° C., preferably by 2.0° C. to 10.0° C., preferably by 3.0° C. to 9.0° C. Preferably, the cooling liquid circulates cocurrentwise with respect to the reaction medium.

Advantageously, the cooling of the reaction medium makes it possible to keep the temperature of the reaction medium within the desired temperature ranges. Any type of heat exchanger known to a person skilled in the art which makes it possible to carry out said process can be used.
According to a first embodiment, at least one heat exchanger(s) is located inside the reaction chamber of the reactor, and is suitable for the cooling of the reaction medium. Preferably, said heat exchanger(s) is (are) positioned longitudinally with respect to said reaction chamber of the reactor; preferentially, a heat exchanger is positioned in each reaction zone and more preferentially still a single heat exchanger is used inside said chamber.
According to a second embodiment, at least one heat exchanger(s) is located outside said reactor and each heat exchanger is incorporated in a recirculation loop comprising withdrawal means and introduction means for introducing the cooled reaction medium into the reaction chamber.

The withdrawal means make it possible, by virtue of a liquid recirculation pump, to send a fraction of the withdrawn liquid phase of the reaction medium to the heat exchanger. Thus, the heat produced by the reaction is removed and the withdrawn liquid is cooled in order to be introduced into said chamber via the introduction means.

For each recirculation loop, the withdrawal of the liquid phase of the reaction medium is carried out starting from a point located below the point of introduction of the cooled liquid into said chamber. For a given reaction zone, the withdrawal is preferably carried out in the lower part of the reaction zone.

For the first reaction zone Z1, in the direction of flow of the liquid phase and of the gas phase, the withdrawal is preferably carried out under the level of introduction of the olefin and preferentially in the bottom of the chamber.

The withdrawal is carried out by any means capable of carrying out the withdrawal and preferably by using a pump.

The liquid phase of the reaction medium of the chamber of the reactor is withdrawn by admission means under the control of the liquid level, so as to keep the latter constant. The admission means are any means well known to a person skilled in the art, such as a valve.

Preferably, the withdrawal flow rate is between 500 and 12 000 t/h and preferably between 800 and 8500 t/h. The withdrawal flow rate is regulated in order to maintain a constant liquid level in the reaction chamber.

For each recirculation loop, the introduction of the cooled liquid into the reaction chamber is carried out starting from a point located above the liquid withdrawal point. For a given reaction zone, the introduction is preferably carried out in the upper part of said reaction zone.

For the final reaction zone Zn of the series, in the direction of flow of the liquid phase and of the gas phase, the introduction is preferably carried out into the gas phase and by any means known to a person skilled in the art. Preferably, the flow rate for introduction of the cooled liquid into the reaction chamber is between 500 and 12 000 t/h and preferably between 800 and 8500 t/h.

Advantageously, carrying out the cooling of the reaction medium via the recirculation loop also makes it possible to carry out the stirring of the medium and thus to homogenize the concentrations of the reactive entities throughout the liquid volume of the chamber of the reactor.

### Stage d) of Recovery of the Reaction Effluent

The process according to the invention comprises a stage d) of recovery of a liquid reaction effluent, in the upper part of the reaction chamber of the reactor, preferably at the top of said chamber. The reaction effluent comprises the desired products, such as linear olefins and more particularly linear α-olefins, the reactants of the reaction (the catalytic system and potentially the olefin introduced) and optionally the solvent and/or the additive.

The catalytic system is advantageously deactivated continuously by any usual means known to a person skilled in the art and then the products resulting from the reaction, and also the solvent, are separated, for example by distillation. The residues of the catalytic system included in a heavy fraction can be incinerated. The olefin which has not been converted can be recycled.

The products resulting from the reaction are preferably linear α-olefins, such as linear olefins comprising from 4 to 12 carbon atoms, preferably from 4 to 8 carbon atoms. Preferably, the linear α-olefins are chosen from but-1-ene, hex-1-ene or oct-1-ene.

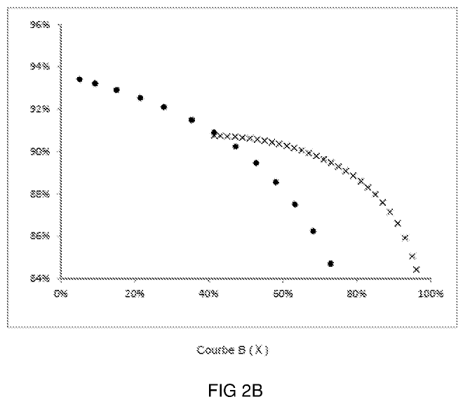

On referring to the curve of FIG. 2B (represented by crosses), it is noteworthy to observe that the process according to the invention makes it possible, under operating conditions equivalent to those of the prior art, to improve the conversion of olefins while retaining a good selectivity for desired products, i.e. for linear α-olefins. There exists an infinity of curves such as the curve of FIG. 2B represented by crosses, according to the point of selectivity chosen for improving the conversion. The profiles of these curves are substantially identical.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application No. 18/58.608, filed Sep. 21, 2018, are incorporated by reference herein.

### EXAMPLES

The examples below illustrate the invention without limiting the scope thereof.

### Example 1 (Comparative)

Example 1 illustrates the reference case corresponding to the FIG. 1, in which the oligomerization process employs a noncompartmentalized stirred gas/liquid reactor according to the prior art.

A mixture of chromium tris(2-ethylhexanoate) (denoted Cr(2-EH)3), of bis(2-(tert-butyl)-6-phenylphenoxy)magnesium and of dibutyl ether (in a 1/1/2 molar ratio) at 0.3 mol/l in a cyclohexane/heptane mixture is prepared in accordance with the protocol described in the patent application FR 3 019 064.

### Implementation of the Process for the Oligomerization of Ethylene According to the Prior Art, at a Pressure of 5.3 MPa and at a Temperature of 135° C., Comprising the Following Stages:

the chromium-based catalytic system composed of Cr(2-EH)3, of bis(2-(tert-butyl)-6-phenylphenoxy)magnesium, of dibutyl ether and of triethylaluminum (Cr/Mg/DBE/Al molar ratio 1/1/2/2.5) is introduced, in the presence of a solvent which is cyclohexane, so as to obtain a content of 5 ppm of chromium, into the liquid phase of the 175 m3 reaction chamber comprising a liquid phase and a gas phase;
said catalytic system is brought into contact with ethylene by introducing the gaseous ethylene into the lower part of said chamber; the residence time in the reaction chamber is 16.43 minutes;
the reaction effluent is recovered.

The volumetric productivity of this reactor is 178 kg of α-olefin produced per hour and per m³ of reaction volume.

The performance qualities of this reactor make it possible to convert 50.80% of the injected ethylene and to achieve a selectivity of 89.50% for the desired α-olefin, for a content by weight of solvent of 3.7. Said content of solvent is calculated as the ratio by weight of the flow rate of injected solvent to the flow rate of injected gaseous ethylene.

### Example 2 (According to the Invention)

Example 2 illustrates the case corresponding to the curve of FIG. 2B (represented by crosses), in which the oligomerization process employs a compartmentalized gas/liquid reactor comprising a reaction chamber, an internal heat exchanger and four perforated plates positioned equidistantly in the liquid height, thus defining five reaction zones. The perforated plates comprise openings with a diameter of 10 mm, occupying 60% of the total surface area of a plate. Each reaction zone is equipped with a heat exchanger which makes it possible to carry out the reaction under substantially isothermal conditions.

The reaction chamber of the reactor measures 3.41 m in diameter, with a liquid height of 20.48 m and a working volume of 188 m³. The H/W ratio is 6.0.

The catalytic composition used is identical to that used in example 1.

### Implementation of the Process for the Oligomerization of Ethylene According to the Invention, at a Pressure of 5.3 MPa and at a Temperature of 135° C., Comprising the Following Stages:

a) the introduction is carried out of the chromium-based catalytic oligomerization system at a chromium content of 5 ppm, in the presence of a solvent which is cyclohexane, into the reaction chamber comprising a liquid phase and a gas phase, and of ethylene, the ethylene being introduced into the lower part of said chamber of the reactor; the residence time in this reactor is 23.62 minutes;
b) said catalytic system is brought in contact with ethylene;
c) the reaction medium is cooled by means of an internal heat exchanger;
d) a reaction effluent is recovered at the top of the reaction chamber.

The volumetric productivity of this reactor is 166 kg of α-olefin produced per hour and per m³ of reaction volume.

The performance qualities of this reactor make it possible to convert 63.98% of the injected ethylene and to achieve a selectivity of 89.77% for the desired α-olefin, for a content by weight of solvent of 3.37. Said content of solvent is calculated as the ratio by weight of the flow rate of injected solvent to the flow rate of injected gaseous ethylene.

For one and the same selectivity for desired α-olefin as in the preceding example, the reactor according to the invention makes it possible to significantly improve the conversion of the ethylene: more than 25% extra conversion.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compartmentalized oligomerization reactor (D) having an upward stream of a liquid phase and of a gas phase forming a reaction medium, said reactor comprising:
a chamber (1) of elongated shape along the vertical axis with a height to width (H/W) ratio between 1 and 8;
means for introduction of a catalytic system (4) and of an olefin (3);
at least one heat exchanger (2) capable of cooling the reaction medium by means of a cooling liquid (6);
a means for recovery (7) of a liquid reaction effluent located in the final reaction zone (Zn) in the direction of flow of the liquid phase and of the gas phase;
a means for bleeding off (5) the gas phase, which gas phase is located at the top of said reactor (D);
characterized in that a plurality of compartmentalization means (8) are located inside the chamber (1) of said reactor (D), each compartmentalization means (8) extending radially over the entire section of the chamber (1) of said reactor, so as to form a plurality of reaction zones (Z1, Z2, . . . , Zn) laid out vertically in tiers, and in that each compartmentalization means (8) comprises a plurality of openings (12) with a diameter between 1 and 20 mm suitable for the passage of the liquid phase and of the gas phase from one reaction zone to the next, said plurality of openings (12) occupying between 3% and 60% of the total surface area of each compartmentalization means (8).

2. The reactor as claimed in claim 1, in which the compartmentalization means are provided in the form of perforated plates.

3. The reactor as claimed in claim 1, in which at least one heat exchanger is located inside the reaction chamber.

4. The reactor as claimed in claim 3, comprising a single heat exchanger extending along the vertical axis of the reaction chamber (1) in each reaction zone (Z1, Z2, . . . , Zn).

5. The reactor as claimed in claim 1, comprising a plurality of heat exchangers (2) located outside the reaction chamber (1), each heat exchanger (2) being associated with a reaction zone (Z1, Z2, . . . , Zn).

6. The reactor as claimed in claim 1, comprising between 2 and 30 reaction zones (Z2, Z3, . . . , Z30).

7. An olefin oligomerization process employing the reactor as claimed in claim 1, at a pressure between 1.0 and 10.0 MPa and at a temperature between 0° C. and 200° C., comprising the following stages:
a) the olefin and a catalytic oligomerization system comprising at least one metal precursor and at least one activating agent are introduced into the liquid phase of the reaction chamber (1);
b) said olefin and said system are brought into contact in each reaction zone (Z1, Z2, . . . , Zn);
c) the reaction medium is cooled by means of at least one heat exchanger (2);
d) a liquid reaction effluent (7) is recovered in the upper part of the reaction chamber of the reactor.

8. The oligomerization process as claimed in claim 7, in which the heat exchanger(s) decrease the temperature of the reaction medium by 1.0 to 11.0° C.

9. The oligomerization process as claimed in claim 7, in which the metal precursor used in the catalytic system is chosen from compounds based on nickel, on titanium or on chromium.

10. The oligomerization process as claimed in claim 7, in which the concentration of nickel, of titanium or of chromium is between 0.01 and 300.0 ppm by weight of atomic metal, with respect to the reaction mass, preferably between 0.02 and 100.0 ppm, more preferably between 0.03 and 50.0 ppm, more preferably between 0.5 and 20.0 ppm and more preferably still between 2.0 and 50.0 ppm by weight of atomic metal, with respect to the reaction mass.

11. The oligomerization process as claimed in claim 7, in which the catalytic system additionally comprises one or more activating agents chosen from aluminum-based compounds, such as methylaluminum dichloride (MeAlCl₂), dichloroethylaluminum (EtAlCl₂), ethylaluminum sesquichloride (Et₃Al₂Cl₃), chlorodiethylaluminum (Et₂AlCl), chlorodiisobutylaluminum (i-Bu₂AlCl), triethylaluminum (AlEt₃), tripropylaluminum (Al(n-Pr)₃), triisobutylaluminum (Al(i-Bu)₃), diethylethoxyaluminum (Et₂AlOEt), methylaluminoxane (MAO), ethylaluminoxane and modified methylaluminoxanes (MMAO).

12. The oligomerization process as claimed in claim 7, in which the olefin is ethylene.

13. The process as claimed in claim 7, in which the linear olefins obtained are linear α-olefins chosen from but-1-ene, hex-1-ene or oct-1-ene.

14. The process as claimed in claim 7, in which the time over which the olefin and the catalytic system are brought into contact in each reaction zone (Z1, Z2, . . . , Zn) is between 1 and 20 seconds.

15. The process as claimed in claim 7, in which the residence time of the reaction medium in said chamber is between 30 and 400 minutes.
